(19) United States
(12) Patent Application Publication
(10) Pub. No.: US 2014/0107790 A1
(43) Pub. Date: Apr. 17, 2014

(54) INTERVERTEBRAL IMPLANT AND INSERTION DEVICE

(71) Applicant: Zbigniew Combrowski, Tuttlingen (DE)

(72) Inventor: Zbigniew Combrowski, Tuttlingen (DE)

(73) Assignee: Z-MEDICAL GMBH & CO. KG, Tuttlingen (DE)

(21) Appl. No.: 14/111,676

(22) PCT Filed: Apr. 13, 2012
(86) PCT No.: PCT/EP2012/056816

§ 371 Date: Dec. 23, 2013

(30) Foreign Application Priority Data
Apr. 15, 2011 (DE) 102011002076.4

(51) Int. Cl. A61F 2/44

(52) U.S. Cl. CPC A61F 2/442
USPC 623/17.16

(57) ABSTRACT

An intervertebral implant (1, 30, 45, 52) with a non-movable bottom plate, a movable bottom plate (2, 31, 46, 54) and a movable top plate (3, 32, 47, 55).

### BACKGROUND OF THE INVENTION

The invention relates to an intervertebral implant and to a device for inserting the intervertebral implant.

There are a great many different indications for stabilization. For example, stabilization procedures are performed in the case of tumors, infections, trauma, and in the treatment of degenerative diseases of the spinal column. In stabilization procedures, one or more segments of the spinal column are surgically connected to each other, e.g. using a system of screws and rods, a system of plates and screws, or a system of hooks or wires.

This stabilization may be non-movable, but it may also be provided with certain dynamics (the connection of the vertebral bodies permits a defined movement).

From biomechanics, it is known that the spinal column as a load-bearing unit can be compared to the statics of a crane.

The front column, which consists of vertebral bodies and intervertebral disks, bears ca. 80% of the load, while the dorsal structures bear ca. 20% in the form of shearing forces.

It can be inferred from this that the anterior segment is exposed mainly to compression forces and the dorsal segment to tensile forces.

In addition, shearing forces, torsional forces and bending forces act on the two columns.

Whereas the abovementioned systems of screws and rods can be used in the dorsal area, ventral support is primarily provided using an intervertebral implant or cage or an implant for replacement of one or more vertebrae.

The ventral support is intended to take place with distraction in order to avoid dislocation of the implant, and it should be attempted, by means of dorsal compression, to obtain a position that is as close as possible to the physiological position in order to maintain the tension band wiring.

This can be achieved by a combination of a ventral and dorsal approach (or in some cases purely ventrally on the cervical spine), or by a dorsal, dorsolateral or lateral approach.

In order to achieve a lasting stability, osseous fusion should be sought, which takes place by bone material growing through and onto the implant and the intervertebral space.

An implant of the correct size is chosen depending on the structures of the spinal column that are found.

Account should be taken of the height and the length of the intervertebral implant.

In order to avoid dislocation of the implant and to achieve a suitable physiological alignment, the previously known and customary cage is driven in with a close fit.

For this purpose, the height that is generally chosen is one that corresponds to the intervertebral space or that is slightly higher, in order to ensure a secure fit.

The cage is inserted via a holding instrument, usually one having a handle with an end which can be struck, if necessary, in order to bring the implant to an end position close to the desired position.

The object of the invention is to make available an intervertebral implant that can be used in a variety of ways and that can be inserted gently and safely and provides an optimal fit, and a device for inserting the intervertebral implant.

### SUMMARY OF THE INVENTION

This object is achieved by providing an intervertebral implant with a non-movable bottom plate, a movable bottom plate and a movable top plate.

Intervertebral implants according to the prior art preferably comprise a non-movable bottom plate and a movable top plate.

The intervertebral implant of the invention preferably comprises a movable bottom plate and a movable top plate. Particularly preferably, the intervertebral implant comprises an adjustment mechanism suitable for moving the bottom plate and the top plate away from each other at the same time and by the same distance. The expression “by the same distance” is intended to refer to an expansion away from an actual center point of the intervertebral implant. The expansion can entail either the entire intervertebral implant or only the bottom plate or the top plate.

The expression “at the same time and by the same distance” covers the following movements of the top plate and of the bottom plate away from each other: a radial expansion, a parallel movement such that the bottom plate and the top plate are adjusted by a distance relative to each other across their entire surface, an adjustment of the height of the intervertebral implant across its entire outer surface.

The movement away from each other at the same time and by the same distance affords the advantage that the intervertebral implant spaces the vertebrae uniformly apart from each other and does not jam. Moreover, it is advantageous that a dislocation of the intervertebral implant is virtually ruled out. There is also the advantage that distraction is not absolutely necessary for inserting the intervertebral implant.

In typical embodiments, the bottom plate is a separate component. The top plate is preferably a separate component. This affords the advantage that the two components can be spaced apart from each other at the same time and by the same distance and do not spread away from each other in a wedge shape.

The bottom plate and the top plate are preferably designed identically and have the same shape. Particularly preferably, the bottom plate and the top plate are designed as half-shells, plates or the like. The surfaces can have a radius, extend parallel to each other and/or enclose an angle to support a lordosis/kyphosis. This affords the advantage that the intervertebral implant can be produced cost-effectively and the direction of installation plays no role.

The adjustment mechanism expediently has a self-locking design. The adjustment mechanism preferably comprises a self-locking thread. This affords the advantage that, when the bottom plate and/or top plate is loaded, the intervertebral implant does not collapse or give way.

The adjustment mechanism preferably comprises an expansion wedge. Particularly preferably, the adjustment mechanism comprises two expansion wedges. More preferably, the expansion wedges are connected by a spindle for adjusting the expansion wedges. This affords the advantage that the intervertebral implant can be adjusted uniformly across its entire outer surface in a simple manner.

The adjustment mechanism and in particular the expansion wedges advantageously have openings and/or holes suitable for filling with bone substitute material, in order to ensure fusion of the implant to the bone.

Particularly preferably, the spindle has a right-handed/left-handed thread. This affords the advantage that the two expansion wedges can be adjusted with one movement at the same time and by the same distance.

In typical embodiments, the expansion wedges are directed toward each other. This affords the advantage that the adjustment mechanism has only a small overall height.

In typical embodiments, the adjustment mechanism comprises at least one column drive. This affords the advantage that the adjustment mechanism is of a very simple design and is protected from contamination. Particularly preferably, the adjustment mechanism comprises two or more column drives.

In typical embodiments, the column drive comprises, on its circumference, a toothing. This affords the advantage that the toothing can be brought into operative connection with a drive spindle which likewise has a toothing. This affords the advantage that the adjustment mechanism can be adjusted very easily with the column drive.

In further embodiments, the adjustment mechanism is designed as a hydraulic or pneumatic system with cushions, in particular made of silicone.

In further embodiments, the intervertebral implant is filled with bone substitute material via a cartridge.

The intervertebral implant preferably comprises an opening for introduction of a bone substitute material. The intervertebral implant can be filled with bone substitute material before and/or after being inserted into the body. This affords the advantage that the intervertebral implant is fixed, more stable and/or secured against slipping. It is also advantageous that a hollow space that has been created in the tissue by the insertion of the intervertebral implant can be filled again. Advantageously, the bone substitute material serves for osseous colonization of the intervertebral implant.

Particularly preferably, the adjustment mechanism comprises the opening for introduction of the bone substitute material. This affords the advantage that the introduction of the bone substitute material can take place after the navigation, adjustment and deployment of the intervertebral implant, even while the latter is firmly fixed in the insertion instrument.

Expediently, at least a proximal end and/or a distal end of the intervertebral implant have/has a conical shape. This affords the advantage that the intervertebral implant can be more easily applied.

The intervertebral implant advantageously comprises, on its surface, a plurality of spikes, ribs, elevations or grooves. This affords the advantage that the intervertebral implant does not slip within the intervertebral disk space.

In typical embodiments, the intervertebral implant has a proximal end in the form of a self-boring tip or a self-boring thread. This affords the advantage that the intervertebral disk space does not have to be excavated for insertion.

The bottom plate and/or the top plate expediently have/has a surface that is a screw shape or thread shape. This affords the advantage that the intervertebral implant is secured against slipping or twisting.

Protection is claimed separately for a device for inserting an intervertebral implant.

The insertion device preferably comprises a drive spindle suitable for actuating the adjustment mechanism of the intervertebral implant. This affords the advantage that the intervertebral implant can be deployed when correctly positioned.

The device preferably comprises a pair of clamping jaws for holding the intervertebral implant. This affords the advantage that the intervertebral implant can be held and moved by the insertion device.

Particularly preferably, the intervertebral implant comprises recesses, drivers or the like that can be brought into engagement with the clamping jaws.

In typical embodiments, the insertion device comprises a pivot mechanism. The pivot mechanism is preferably suitable for pivoting, navigating and orienting the intervertebral implant.

The insertion device expediently comprises a cannula. Particularly preferably, the cannula is suitable for introducing bone substitute material into the intervertebral implant. Particularly preferably, the drive spindle is hollow and forms the cannula. This affords the advantage that the bone substitute material can be introduced directly into the adjustment mechanism of the intervertebral implant.

Preferably, in order to insert an intervertebral implant according to the invention, the intervertebral disk space is firstly excavated and the intervertebral implant is positioned in the installation state using the insertion device. The advantage of intervertebral implants that are self-cutting is that it is possible to dispense with the excavation of the intervertebral disk space.

By actuation of the pivot mechanism of the device, the intervertebral implant can be turned and correctly positioned. When the intervertebral implant is correctly positioned, the drive spindle of the insertion device is used to deploy the intervertebral implant until it sits securely in the intervertebral disk space.

It is possible for the implant to be inserted further into the tissue by a striking surface of the handle of the insertion instrument being struck.

The fact that the intervertebral implant is adjustable also proves advantageous when removing the intervertebral implant. The device for inserting the intervertebral implant can be used to try to collapse the intervertebral implant again in order to remove it from the hollow space that has been created.

The intervertebral implant according to the invention is preferably suitable as a ventral support in combination with a dorsal stabilization system, e.g. a rod/screw system, or as a stand-alone solution.

The intervertebral implant advantageously has a height, width, surface, shape and/or adjustment height or excursion suitable for the intended application.

### BRIEF DESCRIPTION OF THE DRAWINGS

The invention is briefly described below with reference to the attached figures, in which:

FIG. 1 shows a schematic representation of a longitudinal section of an intervertebral implant according to the invention in an installation position;

FIG. 2 shows a schematic representation of a plan view of the intervertebral implant according to the invention from FIG. 2 in the installation position;

FIG. 3 shows a schematic representation of a perspective view of the intervertebral implant according to the invention from FIG. 1 in the installation position;

FIG. 4 shows a schematic representation of a longitudinal section of the intervertebral implant according to the invention from FIG. 1, wherein a bottom plate and a top plate are spaced apart from each other;

FIG. 5 shows a schematic representation of a perspective view of the intervertebral implant from FIG. 4, in which a bottom plate and a top plate are spaced apart from each other;

FIG. 6 shows a schematic representation of a side view of a further embodiment of an intervertebral implant according to the invention;

FIG. 7 shows a schematic representation of a partial section of the intervertebral implant from FIG. 6, with an insertion device;

FIG. 8 shows a schematic representation of a sectional view of a further embodiment of an intervertebral implant according to the invention;

FIG. 9 shows a schematic representation of a perspective view of the intervertebral implant from FIG. 8;

FIG. 10 shows a schematic representation of a longitudinal section of a further embodiment of an intervertebral implant according to the invention, of which the surface has a screw-shaped design;

FIG. 11 shows a schematic representation of a perspective view of the intervertebral implant with screw-shaped outer surface from FIG. 10;

FIG. 12 shows a schematic representation of a side view of a device for inserting an intervertebral implant from FIGS. 6 and 7;

FIG. 13 shows a schematic representation of a longitudinal section of a further embodiment of a device for inserting an intervertebral implant from FIGS. 1 to 5;

FIG. 14 shows a schematic representation of an enlarged detail of FIG. 13;

FIG. 15 shows a schematic representation of a perspective view of the intervertebral implant from FIGS. 13 and 14;

FIG. 16 shows a schematic representation of a perspective view of a detail of the insertion device from FIG. 15;

FIG. 17 shows a schematic representation of an enlarged detail of a device for inserting an intervertebral implant, with an intervertebral implant from FIG. 15, in a further working position.

### DETAILED DESCRIPTION

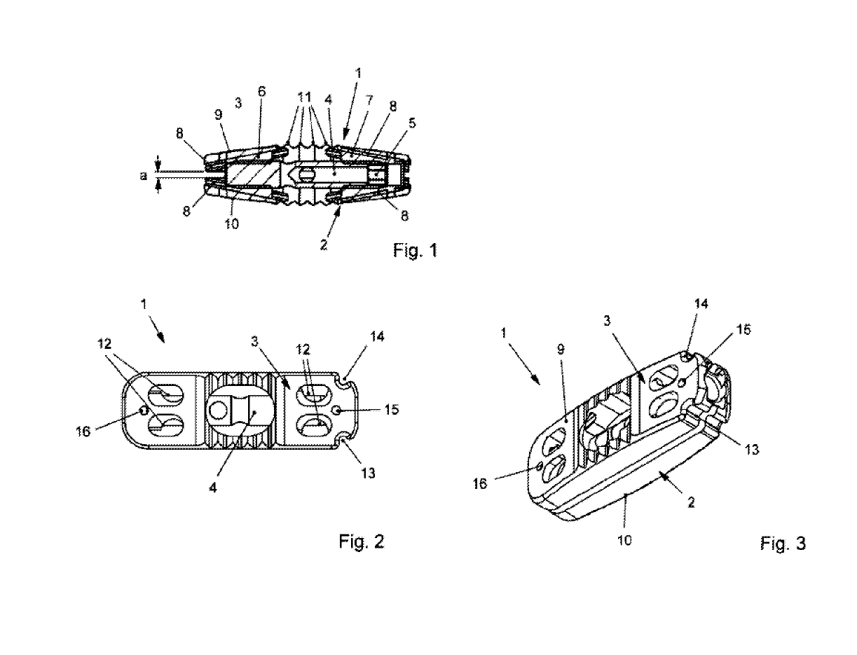

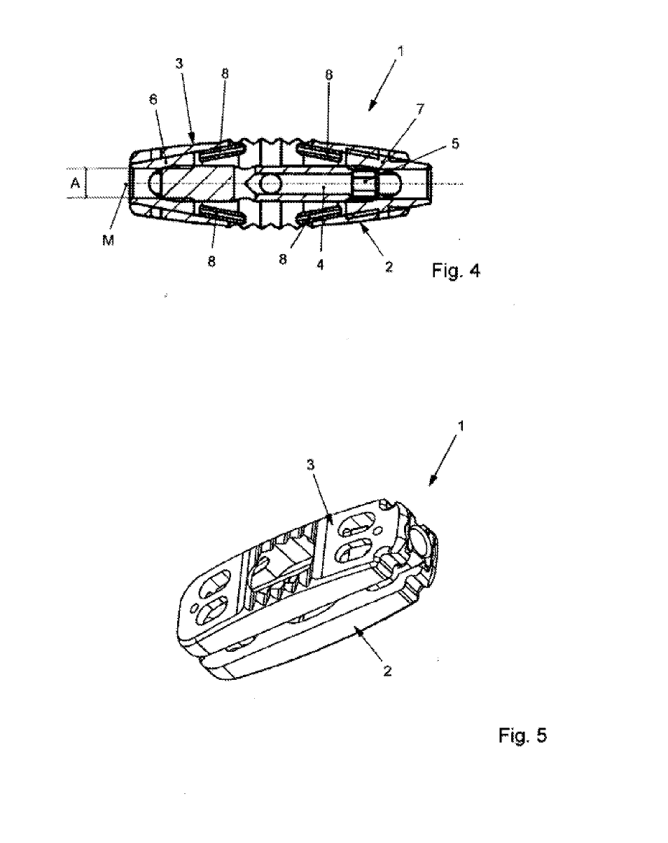

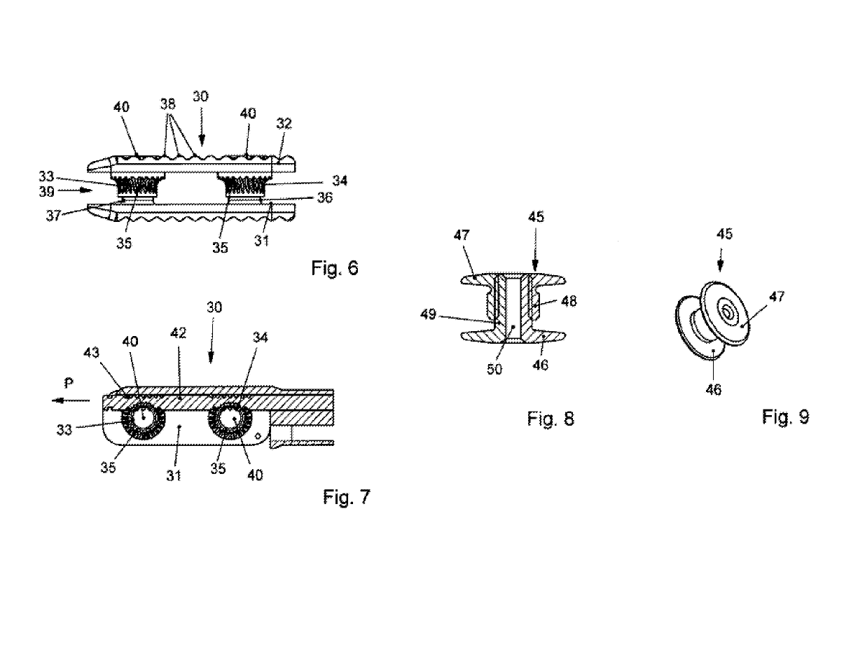

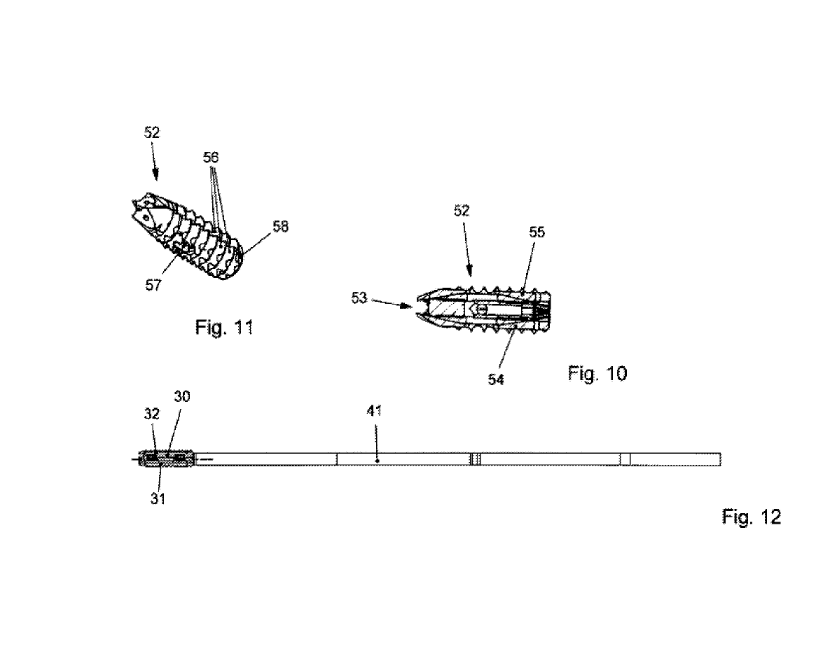

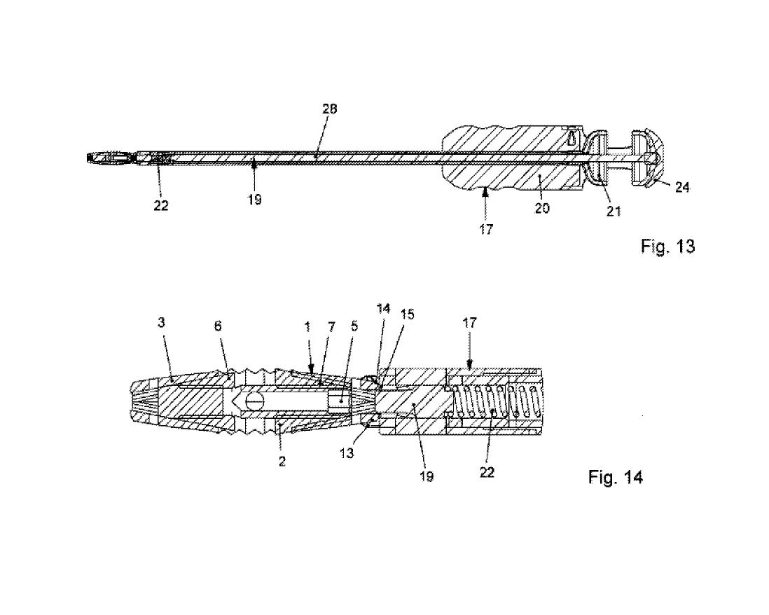

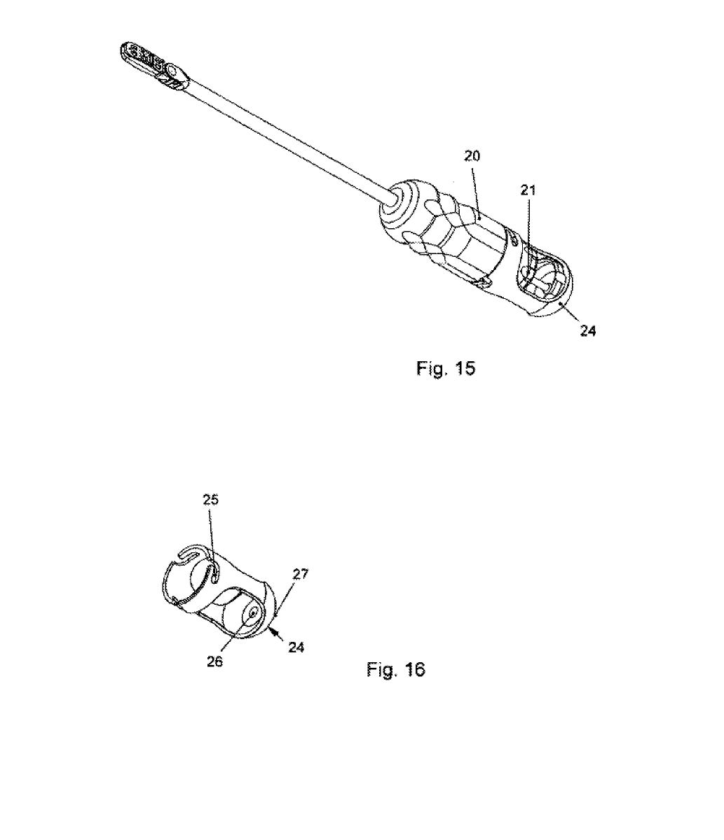

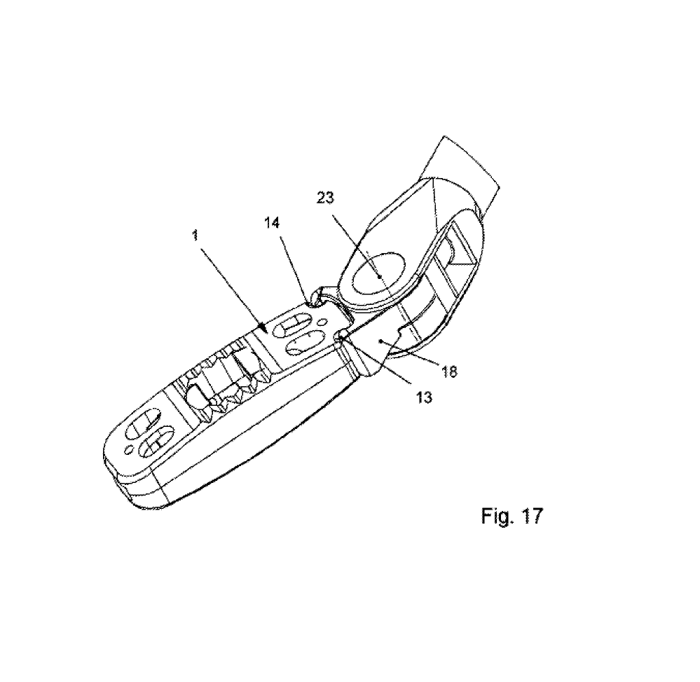

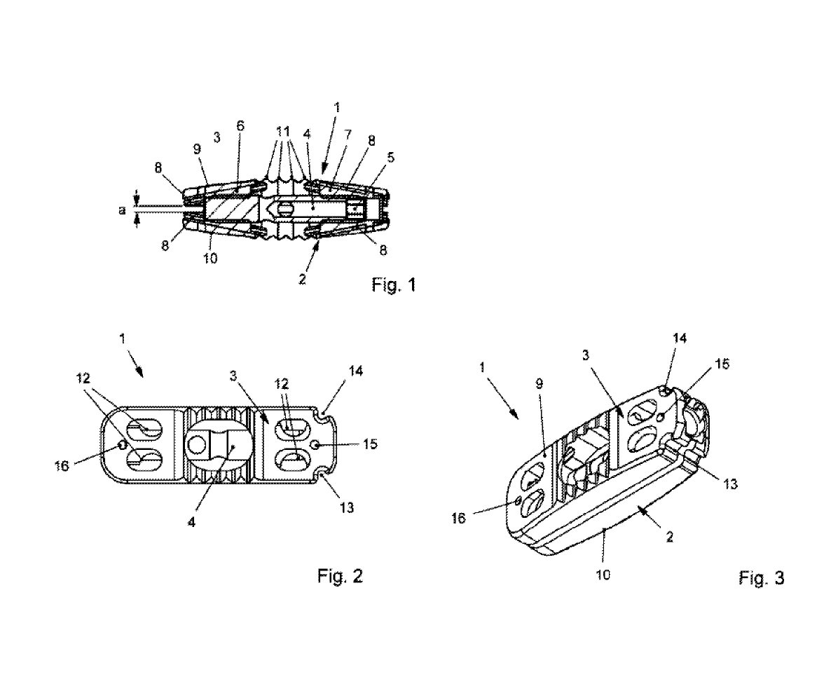

FIG. 1 shows an intervertebral implant 1 according to the invention. The intervertebral implant 1 comprises a bottom plate 2 and a top plate 3. The bottom plate 2 and the top plate 3 are each designed as separate half-shells.

The bottom plate 2 and the top plate 3 receive between them an adjustment mechanism. In the embodiment in FIGS. 1 to 5, the adjustment mechanism comprises a first expansion wedge 6 and a second expansion wedge 7. To better guide the expansion wedges 6 and 7, guide grooves 8 are provided both in the bottom plate 2 and also in the top plate 3. The expansion wedges 6 and 7 are expediently arranged opposite each other, i.e. their ends at which they have a greater height are directed toward each other. This affords the advantage that the overall height of the intervertebral implant can be kept relatively low

Moreover, the adjustment mechanism comprises a spindle 4, as shown in FIGS. 1, 2 and 4. The spindle 4 preferably has a left-handed/right-handed thread for deploying the expansion wedges 6 and 7 at the same time and by the same distance. The spindle 4 preferably comprises a driver 5. The driver 5 is preferably designed as a hexagon stub.

In other embodiments not shown here, the implant comprises only one expansion wedge. Alternatively, it is also possible for two expansion wedges to be arranged in such a way that their tips, facing each other, point to a center of the intervertebral implant.

Moreover, the intervertebral implant 1 has a top surface 9 on the top plate 3 and a bottom surface 10 on the bottom plate 2. They are each designed such that the intervertebral implant 1 has its greatest height in its central area and narrows toward its distal end and toward a proximal end. This affords the advantage that the intervertebral implant 1 can be more easily inserted into an intervertebral disk space and holds securely there.

Moreover, on the surfaces 9 and 10, both the bottom plate 2 and also the top plate 3 have a plurality of elevations 11, such that the intervertebral implant 1 does not slip in the intervertebral disk space.

As is shown in FIG. 2, the intervertebral implant 1 has a plurality of openings 12. The openings 12 are suitable for the introduction of bone substitute material.

In embodiments not shown here, the bone substitute material can also be introduced through the spindle. The openings 12 serve to allow bone substitute material to grow through the intervertebral implant.

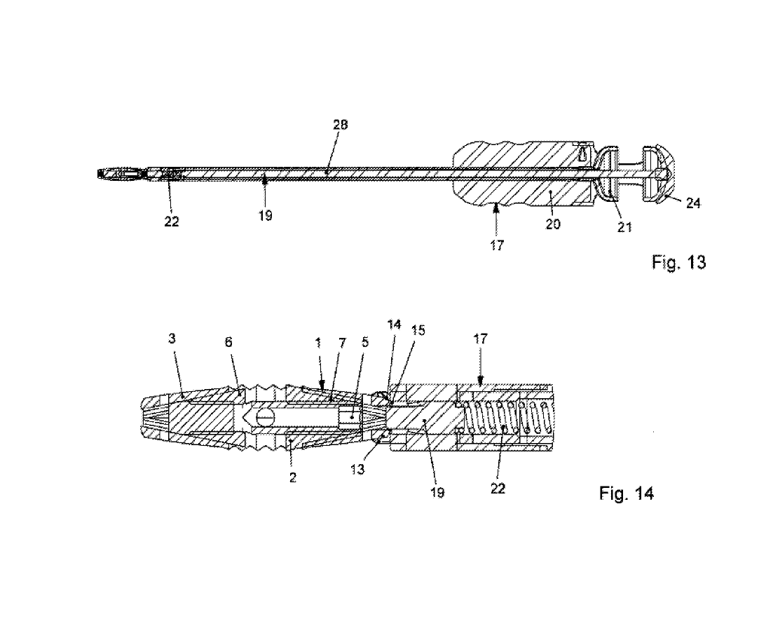

Moreover, at its distal end where the driver 5 is also arranged, the implant as shown in FIGS. 2 and 3 comprises an engagement mechanism for a device 17 for inserting the intervertebral implant 1 according to FIG. 13. As its engagement mechanism, the intervertebral implant has a driver slot 13 and 14 at each of the two corners. As can be seen in FIG. 3, the driver slots 13 and 14 are formed in the corners of the top plate 3 and in the corners of the bottom plate 2. The driver slots 13 and 14 are suitable for the form-fit and force-fit connection to the device 17 for inserting the implant, as can be seen in FIGS. 13 to 15.

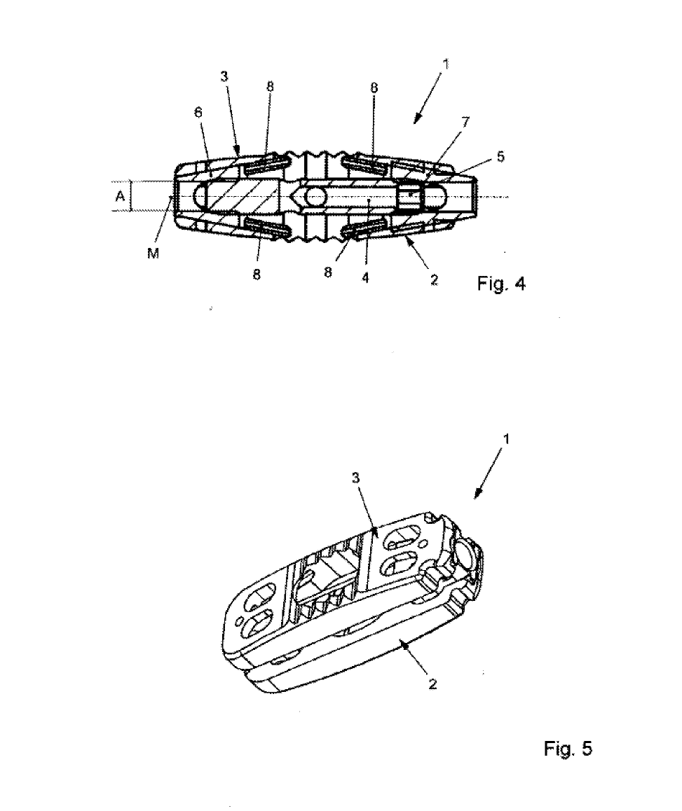

Moreover, the intervertebral implant 1 according to FIGS. 1 to 5 comprises a first spike 15 and a second spike 16. In the present embodiment, the spikes 15 and 16 are fitted in the top plate 3 and serve to hold the intervertebral implant 1 in an intervertebral disk space and to secure it against slipping. Moreover, the spikes 15 and 16 can also be fitted in such a way that, when the bottom plate 2 and the top plate 3 are spaced apart as shown in FIGS. 4 and 5, the spikes are also driven out and anchor the intervertebral implant 1 in the tissue.

The function of the intervertebral implant 1 according to FIGS. 1 to 5 is as follows:

In FIGS. 1 to 3, the intervertebral implant 1 is shown in an installation position. The bottom plate 2 and the top plate 3 are at the smallest possible distance a from each other.

In this installation position with the smallest possible distance a according to FIGS. 1 to 3, the intervertebral implant is inserted into an intervertebral disk space. This affords the advantage that the vertebrae do not have to be drawn apart from each other, as is customary when inserting intervertebral implants according to the prior art.

As is shown in FIGS. 13 to 15, the intervertebral implant 1 is received by the device 17 for inserting the intervertebral implant 1.

The device 17 for inserting the implant 1 has a pair of clamping jaws 15. These engage in the driver slots 13 and 14 of the intervertebral implant 1. Moreover, the device 17 also comprises a drive spindle 19 suitable for engagement in the driver 5 of the intervertebral implant 1.

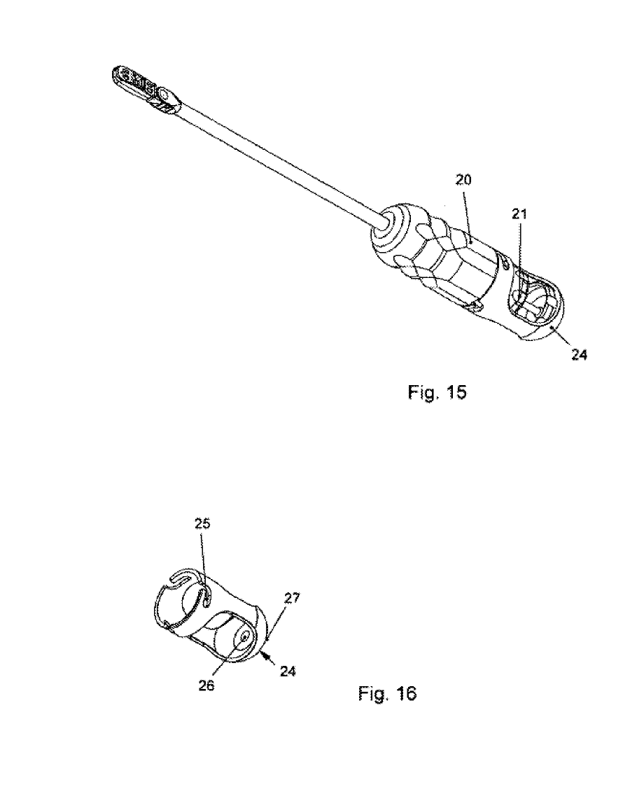

As can be seen in FIG. 13 and FIG. 15, the device 17 also has a handle 20 and a clamping mechanism 21 for controlling the drive spindle 19.

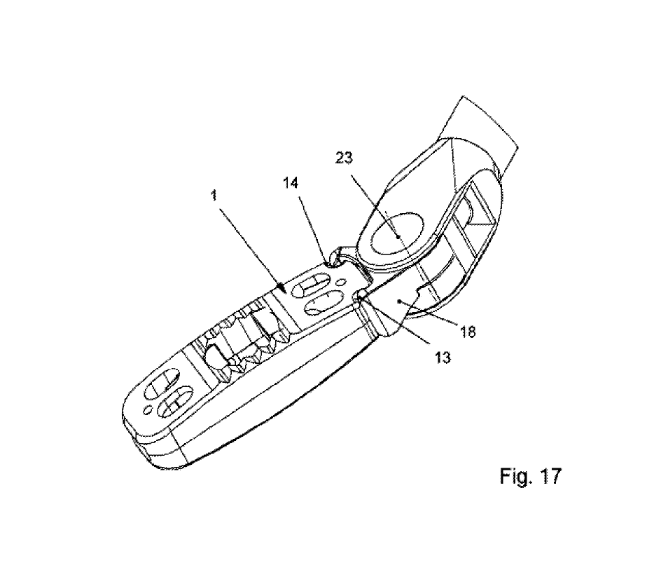

In its front area, the drive spindle 19 comprises a spring element 22. The spring element 22 serves to transmit torque. Moreover, the spring element 22 permits pivoting of the drive spindle 19 about a front hinge 23, as is shown in FIG. 17. The fact that the drive spindle 19 can be pivoted allows navigation and orientation of the intervertebral implant 1 during insertion.

Moreover, the drive spindle 19 comprises a cannula 28 through which bone substitute material can be introduced into the implant.

At its distal end, the device 17 comprises a cap 24. The cap 24 is secured on the handle 20 of the device 17 via a bayonet catch 25. This affords the advantage that the cap 24 can be very quickly connected to the device 17 and also detached from it again. This is particularly advantageous if the cap 24 is damaged.

The cap 24 has a centering point 26 for the drive spindle 19. The centering point 26 is provided on an inside face of the cap 24.

Moreover, the cap 24 has a striking surface 27. The striking surface 27 is provided on a rear face of the cap 24. The striking surface 27 serves for applying impacts when inserting the intervertebral implant 1.

Actuation of the handle 20 and of the clamping mechanism 21 of the device 17 orients the intervertebral implant 1 and positions it in an intervertebral disk space. If necessary, the intervertebral implant 1 can be driven further into the intervertebral disk space by impacts applied to the striking surface 27 of the device 17.

The expansion wedges 6 and 7 of the intervertebral implant 1 are spread open via the drive spindle 19, which engages in the driver 5 of the spindle 4. In this way, the bottom plate 2 and the top plate 3 are spaced apart from each other at the same time and by the same distance, such that their lower edges, directed toward a center axis M, are at a uniform distance A from each other. The bottom plate and the top plate are spaced apart parallel to each other.

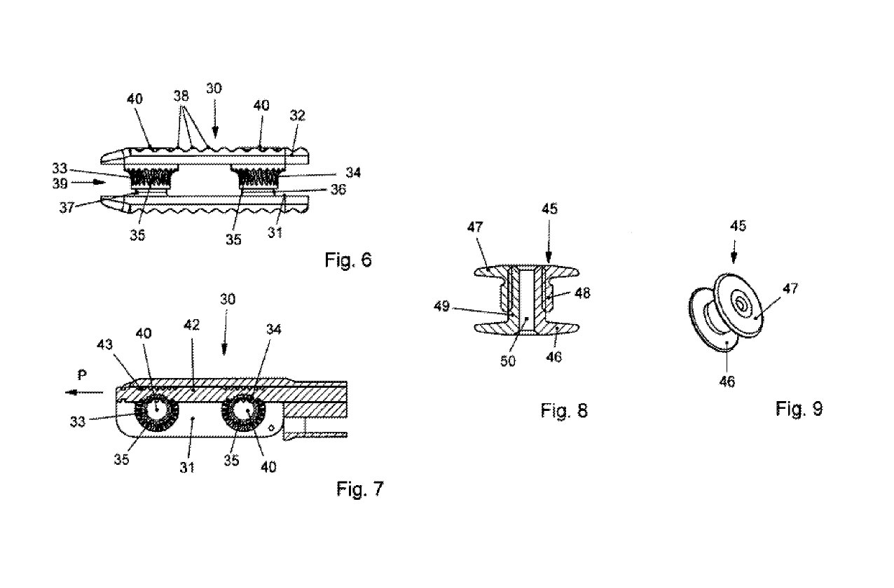

FIGS. 6 and 7 disclose a further embodiment of an intervertebral implant 30. The intervertebral implant 30 comprises a bottom plate 31 and a top plate 32.

The bottom plate 31 and the top plate 32 are connected to each other via a two-column drive. For this purpose, two sleeves 33 and 34 are fitted rotatably into the top plate 32. Each of the sleeves 33 and 34 has a toothing 35 on its circumference. The toothing is the engagement mechanism of the intervertebral implant 30. On their inner sides, the sleeves 33 and 34 each have a thread (not shown).

A first column 36 and a second column 37, which are connected to the bottom plate 31, engage in the sleeves 33 and 34 of the top plate 33. On their outer surfaces, the columns 36 and 37 each have a thread (not shown), which can be brought into operative connection with the threads of the sleeves 33 and 34.

In further embodiments not shown here, the columns can also be fitted rotatably in the intervertebral implant.

The surfaces of the top plate 32 and of the bottom plate 32 are designed substantially analogously to each other. The surfaces preferably extend substantially parallel to each other. As in the embodiment in FIGS. 1 to 5, the intervertebral implant 30 has, on its surface, a plurality of elevations 38. These secure the intervertebral implant 30 against slipping in the intervertebral disk space.

A proximal end 39 of the intervertebral implant 30 has a conical shape. This affords the advantage that the intervertebral implant 30 can be more easily inserted into an intervertebral disk space and holds securely there.

Openings 40 formed in the sleeves 33 and/or 34, and passing through an upper side of the top plate 32, are preferably suitable for introduction of bone substitute material.

The way in which the intervertebral implant 30 functions is as follows:

In the installation state as shown in FIG. 6, the intervertebral implant 30 is inserted, with its proximal end 39 to the front, into an intervertebral disk space. For this purpose, as shown in FIG. 12, the intervertebral implant 30 is fitted on a device 41 for inserting the implant 41.

As is shown in FIGS. 7 and 12, the device 41 engages with a drive spindle 42 between the bottom plate 31 and the top plate 32 of the implant 30.

As can be seen in FIG. 7, the drive spindle 42 of the device 41 has a toothing 43. The toothing 43 of the drive spindle 42 is suitable for engaging in the toothing 35 of the sleeves 33 and 34. By means of a rotating movement of the drive spindle 42, or a movement of the drive spindle 42 in arrow direction P, the bottom plate 31 and the top plate 32 of the intervertebral implant 30 are driven apart from each other. The bottom plate 31 and the top plate 32 are driven apart in parallel, such that they are at the same distance from each other across their entire outer surface.

FIGS. 8 and 9 disclose a further embodiment of an intervertebral implant 45. The intervertebral implant 45 comprises a bottom plate 46 and a top plate 47. Analogously to the previous embodiment in FIGS. 6 and 7, the top plate 47 comprises a sleeve 48, which is in operative connection with a column 49 of the bottom plate 46.

Both the column 49 and also the sleeve 48 have a bore 50, which is suitable for the introduction of bone material or bone substitute material.

The adjustment of the bottom plate 46 relative to the top plate 47 takes place analogously to the embodiment in FIGS. 6 and 7, with the difference that only one column drive is present.

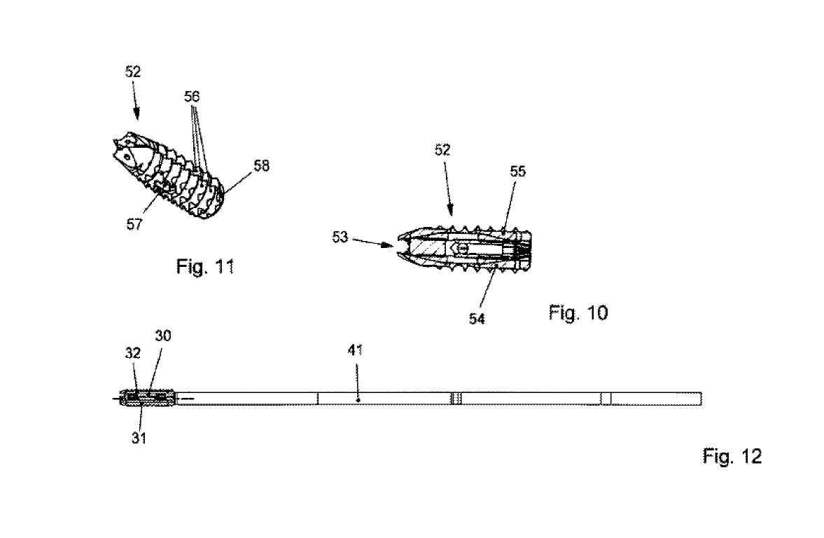

FIGS. 10 and 11 disclose a further embodiment of an intervertebral implant 52. The intervertebral implant 52 is designed substantially analogously to the intervertebral implant 1 in FIGS. 1 to 5.

The adjustment mechanism of the intervertebral implant is designed analogously to the adjustment mechanism of the intervertebral implant 1.

The intervertebral implant 52 is designed to taper conically only at its proximal end 53.

A bottom plate 54 of the intervertebral implant 52 is designed as a half-shell. A top plate 55 of the intervertebral implant 52 is designed as a half-shell. The intervertebral implant 52 thus has the shape of a cigar.

On their surfaces, the bottom plate 54 and the top plate 55 have grooves 56. The grooves 56 are preferably arranged in a helical shape. The grooves 56 prevent twisting of the intervertebral implant 52.

The proximal end 53 of the intervertebral implant 52 is self-cutting or self-boring. This affords the advantage that the intervertebral disk space does not have to be excavated in order to insert the intervertebral implant 52.

Analogously to the intervertebral implant 1, the intervertebral implant 52 also comprises openings 57, which are suitable for the introduction of bone substitute material.

Moreover, the intervertebral implant 52 has driver slots 58. These serve as engagement points for the clamping jaws 15 of the insertion device 17. The insertion device 17 can be used with the intervertebral implant 52 in the same way as with the intervertebral implant 1.

1. An intervertebral implant (1, 30, 45, 52) comprising a non-movable bottom plate, a movable bottom plate (2, 31, 46, 54) and a movable top plate (3, 32, 47, 55).

2. The intervertebral implant as claimed in claim 1, further comprising an adjustment mechanism for spacing the movable bottom plate (2, 31, 46, 54) and the top plate (3, 32, 47, 55) away from each other at the same time and by the same distance.

3. The intervertebral implant as claimed in claim 2, wherein the bottom plate (2, 31, 46, 54) is a separate component.

4. The intervertebral implant as claimed in claim 3, wherein the top plate (3, 32, 47, 55) is a separate component, which is designed substantially like the bottom plate (2, 31, 46, 54).

5. The intervertebral implant as claimed in claim 2, wherein the adjustment mechanism comprises two expansion wedges (6, 7), with a spindle (4) for adjusting the expansion wedges (6, 7).

6. The intervertebral implant as claimed in claim 2, wherein the adjustment mechanism comprises at least one column drive having a sleeve (33, 34, 48) and with a column (36, 37, 49).

7. The intervertebral implant as claimed in claim 6, wherein the column drive has, on a circumference, a toothing (35) as an engagement point for a device for inserting the intervertebral implant.

8. The intervertebral implant as claimed in claim 2, further including an opening (12, 40, 50) for the introduction of bone substitute material.

9. The intervertebral implant as claimed in claim 2, wherein the adjustment mechanism comprises an opening (12, 40, 50) for the introduction of bone material and/or substitute material.

10. The intervertebral implant as claimed in claim 1, wherein at least one of a proximal end (39, 53) and a distal end of the intervertebral implant (1, 30, 52) has a conical shape.

11. The intervertebral implant as claimed in claim 1, wherein the movable bottom plate (54) and the movable top plate (55) have a surface and/or a proximal end (53), which surface and/or end is a self-cutting thread.

12. A device (17, 41) for inserting an intervertebral implant (1, 30, 45, 52) comprising a non-movable bottom plate, a movable bottom plate (2, 31, 46, 54) and a movable top plate (3, 32, 47, 55), further comprising an adjustment mechanism for spacing the movable bottom plate (2, 31, 46, 54) and the top plate (3, 32, 47, 55) away from each other at the same time and by the same distance, including providing a drive spindle (19, 42) and actuating the adjustment mechanism of the intervertebral implant (1, 30, 45, 52) with the drive spindle.

13. The device as claimed in claim 12, including providing a pair of clamping jaws (15) for holding the intervertebral implant (1, 52).

14. The device as claimed in claim 12, including providing a pivot mechanism (23) suitable for pivoting the intervertebral implant (1, 52).

15. The device as claimed in claim 12, including providing a cannula in the drive spindle (19, 42) for filling the intervertebral implant (1, 30, 45, 52) with bone substitute material.
